(19) United States
(12) Patent Application Publication
(10) Pub. No.: US 2010/0047301 A1
(43) Pub. Date: Feb. 25, 2010

(54) Dry Nail Polish Applique Delivery System

(76) Inventor: Fa Young Park, Leonia, NJ (US)

Correspondence Address: LEVISOHN BERGER, LLP, Suite 615, 11 Broadway, New York, NY, 10004, US

(21) Appl. No.: 12/615,410

(22) Filed: Nov. 10, 2009

Related U.S. Application Data
(63) Continuation-in-part of application No. 12/183,385, filed on Jul. 31, 2008.
Application No. 12/183,385 is a continuation-in-part of application No. 12/138,701, filed on Jun. 13, 2008.
Application No. 12/138,701 is a continuation-in-part of application No. 11/866,678, filed on Oct. 3, 2007.
Application No. 11/866,678 is a continuation-in-part of application No. 11/543,481, filed on Oct. 5, 2006.
Application No. 11/543,481 is a continuation-in-part of application No. 11/126,862, filed on May 11, 2005.
(60) Provisional application No. 60/570,713, filed on May 12, 2004.

(51) Int. Cl. A61K 8/02

(52) U.S. Cl. 424/401; 424/59

(57) ABSTRACT

A nail appliqué product for delivering nail additives to a fingernail. The appliqué has an adhesive layer that, in use, is in direct contact with a wearer's fingernail. The adhesive layer contains additives such as vitamins, minerals and/or drugs that are delivered to a fingernail after the appliqué is affixed thereto. The appliqué is flexible and conformable to a fingernail and it continues to deliver additive for an extended period of time.

### RELATED APPLICATIONS

The current application is a Continuation-In-Part of pending U.S. patent application Ser. No. 12/183,385 filed on Jul. 31, 2008, which itself is a Continuation-In-Part of pending U.S. patent application Ser. No. 12/138,701 filed on Jun. 13, 2008, which is a Continuation-In-Part of pending U.S. patent application Ser. No. 11/866,678 filed on Oct. 3, 2007, which is a Continuation-In-Part of pending U.S. patent application Ser. No. 11/543,481 filed Oct. 5, 2006, which is itself a Continuation-In-Part of U.S. patent application Ser. No. 11/126,862, filed on May 11, 2005, which claims domestic priority from U.S. Provisional Patent Application No. 60/570,713, filed on May 12, 2004. The contents of each of the above-mentioned patent applications are incorporated by reference herein.

### FIELD OF THE INVENTION

The current invention relates generally to the field of nail polish, more specifically, to a novel method and apparatus for delivering additive ingredients to fingernails.

### BACKGROUND OF THE INVENTION

The use of an instant fingernail coating product whereby nail polish is applied to a fingernail by adhesively securing to it a dry form of nail polish has become a preferred mode of attaining a manicure by many users. Such dry nail polish products are disclosed in U.S. Pat. Nos. 4,903,840 and 5,415,903 each of which is incorporated by reference herein.

The nail coating product, or instant nail polish referenced above, confers numerous advantages over conventional, prior art nail polish applied with a blush in a liquid form. The application of instant nail polish is faster, easier and cleaner than conventional nail polish and does not suffer from the numerous encumbrances associated with wet nail polish such as drying time and the concern of smearing or smudging.

In addition to adorning fingernails and toenails, many nail polish formulations contain additives such as vitamins, minerals and the like to enhance the health of fingernails.

The current invention is directed to a dry nail appliqué that is also a delivery system of nail polish additives. One advantage of the inventive delivery system is that ingredients to be delivered continue to be delivered well after the initial application thereof and a virtually unlimited variety of additives may be utilized.

### SUMMARY OF THE INVENTION

In the current invention any one of the nail appliqués described in the above-incorporated U.S. patent applications is modified such that the adhesive layer of the appliqué is provided with an added ingredient that is to be delivered to a fingernail.

The inventive nail appliqué comprises one or more layers of nail enamel and an adhesive layer containing one or more ingredients to be delivered to a fingernail. Nail appliqués are roughly sized and shaped to cover a fingernail.

### BRIEF DESCRIPTION Of THE DRAWINGS

FIG. 1 is a bottom perspective view of a slot coating die used in the inventive method.

FIG. 2 front bottom perspective view of the slot coating die of FIG. 1.

FIG. 3 is an elevational view of a shim and half of the slot coating die of FIG. 1 dissembled.

FIG. 4 is an elevational view of the shim and die half of FIG. 3 assembled.

FIG. 5 is a front perspective view of a coating apparatus used in the inventive method.

FIG. 6 is a rear perspective view of the coating apparatus of FIG. 5.

FIG. 7 is a schematic view of a set of nail appliqués.

### DETAILED DESCRIPTION OF THE INVENTION

The following is a detailed description of the preferred embodiments of the invention, reference being made to the drawings in which the same reference numerals identify the same elements of structure in each of the several figures. It should be noted that these drawings are merely exemplary in nature and in no way serve to limit the scope of the invention, which is defined by the claims appearing herein below.

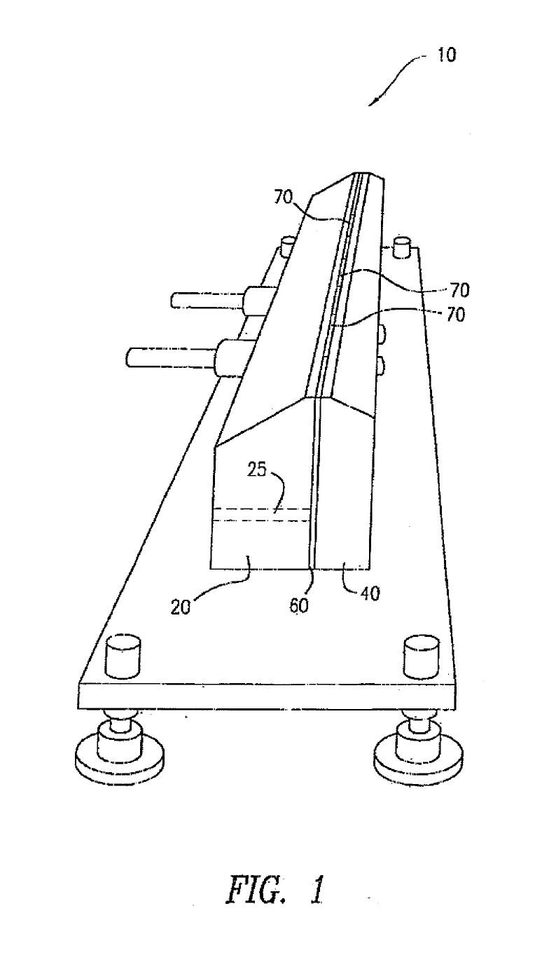

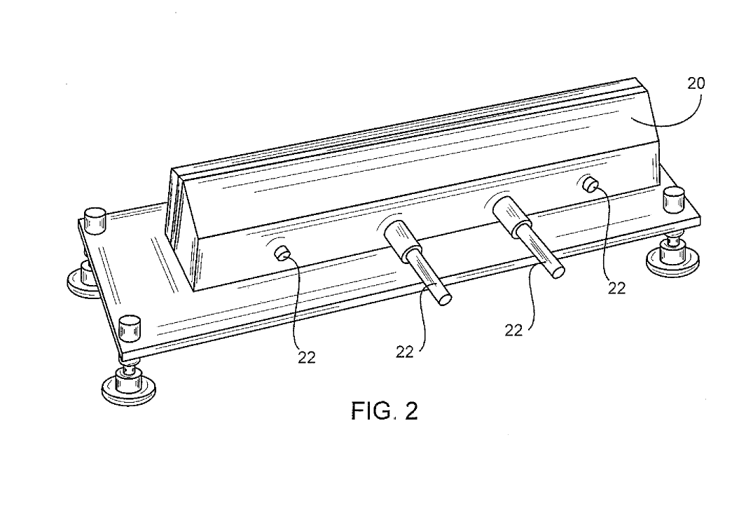

The various coatings of the product are applied via a technique referred to herein as “slot curtain die coating.” The die in question is shown in FIGS. 1-4 in various states of assembly as die 10. As best shown in FIG. 1, die 10 includes front die section 20, rear die section 40, and a specially shaped shim 60 disposed therebetween. All three parts are tightly secured together, preferably by bolting, e g, by bolts 24 (see FIG. 6). Referring to FIG. 2, front die section 20 includes inlets 22 which feed internal boles 25 with liquid nail enamel or any of the other components of the product, such as for example adhesive and additive or a top, clear layer.

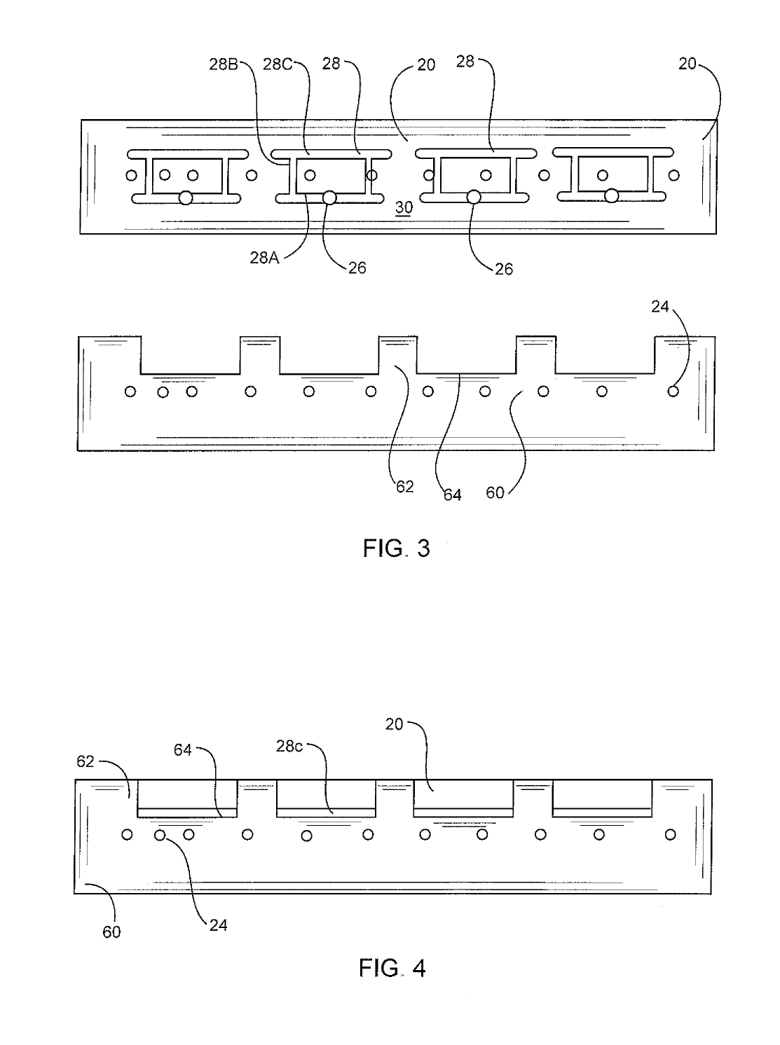

FIGS. 3 and 4 illustrate the interior of die 10; in both of these figures, rear die section 40 has been removed for clarity. Internal bores 25 of front die section 20 terminate in outlet holes 26 on inner face 30 and reside in flow channels 28 thereon. The purpose of flow channels 28 is to direct the liquid nail enamel from outlet holes 26 in a manner that results in consistent and even application of the enamel on the substrate. As such, each flow channel 28 includes upper substantially horizontal branch 28A, which feeds into substantially vertical branches 28B and thence into lower substantially horizontal branch 28C. It should be noted that die 10 is shown in FIGS. 1-4 upside down; hence, fluid exiting outlet hole 26 seeps along horizontal branch 28A, down vertical branches 28B, and then seeps into horizontal branch 28C. The liquid enamel seeps from branch 28C and onto the substrate.

Without shim 60, the two inner faces of front and rear die sections 20 and 40 would be firmly abutting and would not allow room for the enamel to seep out of horizontal branch 28C. However, as shown in FIGS. 3 and 4, shim 60 includes vertical projections 62 between cutouts 64. When shim 60 is attached to front die section 20 by bolts 24 (see FIG. 4), it shields and covers all of flow channel 28 except for the majority of lower horizontal branch 28C. This way, enamel flowing in branches 28A or 28B cannot seep out of these branches but must instead move forward (downward) ultimately to branch 28C. Because branch 28C is uncovered, enamel simply spills out of it and thus out of slots 70 (see FIG. 1) and onto the substrate in a sheet-like or curtain-like configuration.

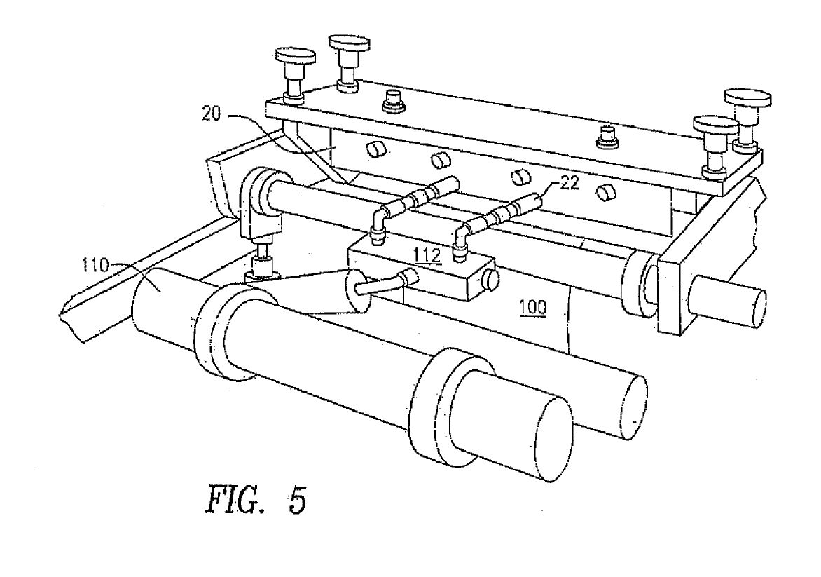

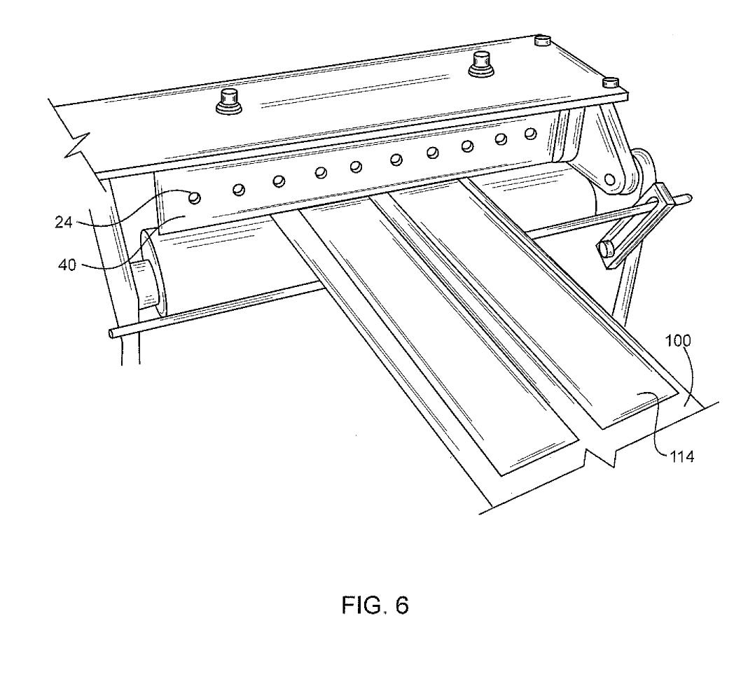

More specifically, as best illustrated in FIGS. 5 and 6, substrate 100 is fed into the machinery by rollers 110. Liquid enamel source 112 is attached to inlets 22 so that heated, pressurized liquid enamel can be forced into die 10. When substrate 100 passes under die 10, liquid enamel or other components being coated, fall out of slots 70 and onto substrate 100 thereby forming layer 114.

The first substance to be applied to substrate 100 is an adhesive material. Nail enamel layers ate applied atop of the adhesive layer. The adhesive layer secures the appliqúe to a fingernail. Because the adhesive layer is in direct contact with a wearer's fingernail, it provides a unique opportunity to act as a delivery vehicle of additives.

It should be noted that in other products that contain additives, the additives are mixed into the nail enamel to form a uniform mixture. This minimizes the amount of additives that could be added and inhibits the efficacy of the same. In the current invention, however, there is a specific zone (the adhesive layer) in which additives are contained. This allows the nail enamel layers to remain free of additives and allows for maximum delivery of the same In some embodiments, additives could be added to the enamel layers as well.

In some embodiments adhesive material and additive are applied to a substrate in separate steps. For example, an additive could first be applied and adhesive substance atop thereto (or vice versa). However, in a preferred embodiment, adhesive material is combined with additives first. The combination is then applied to a substrate.

Examples of additives include, but are not limited to, vitamins, minerals, drugs, and nail additives As non-limiting examples, additives such as Vitamin A, forms of algae, kelp, bamboo, white tea, green tea, papaya fruit, cactus, garlic, protein, calcium etc. may be added to the adhesive layer of an appliqué.

After the adhesive is applied, one or more layers of liquid nail is applied atop thereto. In some embodiments one or more clear layers are applied atop of the nail enamel layers essentially the same manner as described above.

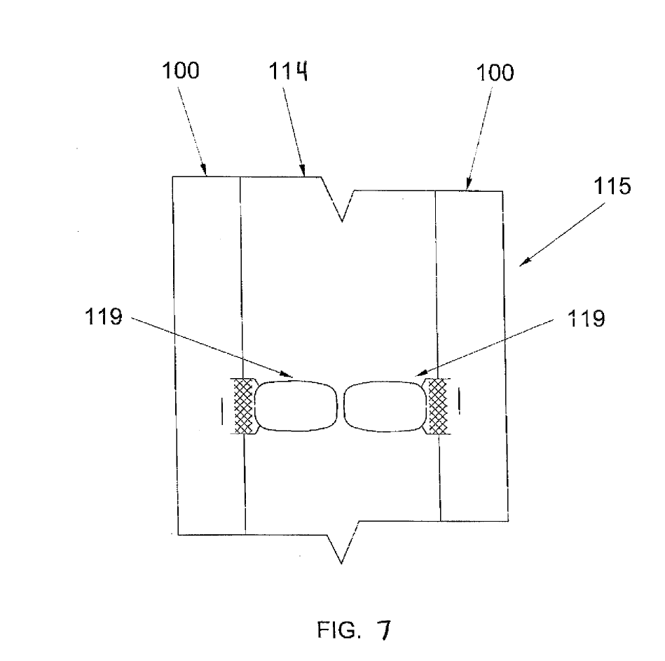

After all layers are applied to the substrate, the substrate is cut into several nail-shaped appliqués. In FIG. 7, a sheet 115 having a strip of nail enamel 114 is shown from which individual appliqués 119 are cut.

The appliqués are partially dried before being sealed in packaging. Once a wearer applies an appliqué, the enamel finishes curing on her fingernail. The adhesive remains in constant contact with the fingernail and continuously delivers additives thereto.

In some preferred embodiments, the inventive nail enamel includes a much higher solids content (35% and up) and/or higher viscosity nitrocellulose (60-80 second and up) than conventional nail polish. These characteristics cannot be used in conventional nail polish because the resultant polish would be too thick (i.e. it would have too high a viscosity) to apply by brush. From a mass manufacturing point of view, however, the less volatile solvent in the formulation, the greater the production capacities. In addition, higher viscosity nitrocellulose (60-80 second) can produce thinner but stronger and shinier film. The multi-layer film has great flexibility in manufacture and can provide a variety of different products.

Three examples of the basic composition of high viscosity liquid raw nail polish for processing and product of semi-dry or dry (hereinafter “semi/dry”) nail polish film follow.

### EXAMPLE 1

A non-metallic dry nail polish film of the present invention uses 35-60% solids, of which 25-35% (w/w) is ¼- and ½-second nitrocellulose. In contrast, conventional bottled liquid nail polishes contain, at most, 13-17% nitnocellulose. The present invention thus doubles the solid content. The present invention includes about 40-50% solvents, as opposed to approximately 70% of solvent used in traditional liquid nail enamels This lower solvent content has several advantages From the standpoint of processing, the time required to complete drying/evaporation (i.e., to produce a finished product) is about 30-40% less than currently available liquid formulations. Second, the dry nail polish film of the present invention is better for the environment and energy saving for oxidate solvents. This nail polish formulation is impossible to use with a brush because it is too thick; however, in the present invention, the formulation is heated to about 100-150 degree F., thereby reducing the viscosity and allowing the material to flow through the nozzle. The first, non-metallic formulation is as follows:

25-35% ¼ or ½ second nitrocellulose
8-12% Polymer, co-polymer resin(s) (e.g. acrylic, polyester, Polyurethane, etc.)

### 8-17% Plasticizer

7-12% color pigments

### 18-25% Ethyl Acetate

### 18-25% Butyl Acetate

(total solids 35-60%)
This formulation is approximately 1500-4000 centipoise (60 rpm) at room temperature

### EXAMPLE 2

As mentioned above, high viscosity nitrocellulose (60-80 second) is conventionally used in less than 1-5% amounts solely for the purpose of adjusting enamel viscosity using less than 5% for bottled chrome nail polish. One manufacturer of such a formulation is Kirker Enterprises in New Jersey, as described in U.S. Pat. No. 6,565,835 to Socci et al. By contrast, the inventive metallic or non-metallic formulation contains high viscosity nitrocellulose (60-80 second) in quantities greater than 6%, up to 25% By using such a high percentage of extremely viscous nitrocellulose, thinner, shinier films with greater strength and flexibility are possible This formulation is as follows:

6-25% 60-80 second nitrocellulose 8-12% polymer, co-polymer resin(s) 5-10% color pigments 4-15% plasticizer 1-2% other solids remainder solvent(s) (e.g, ethyl and butyl acetates, isopropyl alcohol)

This formulation is approximately 1500-4000 centipoise (60 rpm) at loom temperature.

### EXAMPLE 3

A third formulation combines the “best of both worlds” of the first two mentioned above Specifically, the composition of this formulation includes both high viscosity nitrocellulose (60-80 sec.) and ¼ or ½-second nitocellulose in a 40%-60% combination (with respect to each other) This formulation achieves a thinner film with medium strength and flexibility as well as shine:

8-17% ¼ or ½-second nitrocellulose 6-15% 60-80 second nitrocellulose 8-12% polymer, copolymer resin(s) 5-10% color pigments 4-15% plasticizer 1-2% other solids remainder solvent(s) (e.g., ethyl and butyl acetates, isopropyl alcohol)

This formulation is also approximately 1500-4000 centipoise (60 rpm) at room temperature.

In all three examples given above, the differences and benefits of the new inventive formulations for semi/dry nail enamels as compared to conventional liquid nail polish are manifold They produce a stronger film on the nails which lasts much longer than either conventional nail polish or conventional semi/dry nail enamel appliqués. The film is also shinier than those previously produced. The inventive film appliqués are thinner than either conventional salon nail polish jobs or prior appliqués, thereby allowing the nail more breathability. The films are also flexible and may be easily stretched to cover a nail mote fully and completely than before with less solvent remaining (less than 5%). Many different types of films can be produced without significant retooling of the machinery. Finally, since there is a much greater percentage of solids, more film can be produced faster and less expensively.

In conventional coating processes for manufacturing dry nail enamel films, the nitrocellulose base must be of sufficiently low viscosity to flow through very small apertures (i.e., slots and holes of less than 300 microns in slot) in the coating die. Because nail polish formulations (with nitrocellulose bases) having a viscosity of greater than 1000 centipoise generally will not flow readily and would quickly clog the die (especially those containing glitter or large particle mica), 60-80 second nitrocellulose and the like are typically not used in the manufacture of nail polishes, other than in small amounts (e.g, up to a maximum of 5%, typically from 1-3%, for adjusting viscosity of the final product as mentioned above). In one embodiment, the formulation is heated to between 100-150° F., preferably about 125° F., higher viscosity nitrocellulose may be pumped and used. Similarly, where the content of ¼ or ½-second nitrocellulose is greater than about 35% by weight of the composition, then the formulation may be heated to about 100° F., preferably to about 125° F.

Note that the above three examples are directed to the enamel portion of an appliqué—excluding the adhesive portion thereof.

Having described this invention with regard to specific embodiments, it is to be understood that the description is not meant as a limitation since further modifications and variations may be apparent or may suggest themselves to those skilled in the art. It is intended that the present application covet all such modifications and variation as fall within the scope of the appended claims.

What is claimed is:

1. A self adhesive nail appliqué product comprising:
a substrate for carrying a nail enamel appliqué;
a nail enamel appliqué disposed on said substrate wherein said nail appliqué comprises an adhesive layer, said adhesive layer comprising an additive to be delivered to a nail; and
at least one layer of nail enamel disposed on top of said adhesive layer.

2. The product of claim 1, wherein said additive is a vitamin.

3. The product of claim 1, wherein said additive is a mineral.

4. The product of claim 1, wherein said additive is a drug.

5. A method of delivering nail additives to a nail comprising the steps of:
combining an adhesive material and a nail additive;
adding said combination of adhesive material and additive to a nail appliqué; and
applying said nail appliqué to a nail

6. The method of claim 5, wherein said additive is selected from the group consisting of a vitamin, mineral and a drug.
